(19) United States
(12) Patent Application Publication
(10) Pub. No.: US 2007/0004027 A1
(43) Pub. Date: Jan. 4, 2007

(54) Method for manufacturing a biosensor element

(76) Inventors: Miwako Nakahara, Tokyo (JP); Takashi Inoue, Yokohama (JP); Tomonori Saeki, Yokosuka (JP); Osamu Kogi, Yokohama (JP); Noriko Ban, Fujisawa (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/270,456

(22) Filed: Nov. 10, 2005

(30) Foreign Application Priority Data
Jun. 27, 2005 (JP) 2005-186164

(51) Int. Cl. C12M 1/34

(52) U.S. Cl. 435/287.2; 427/2.11; 977/924

(57) ABSTRACT

When probe biomolecules are immobilized on a substrate surface, a surfactant (phase transfer catalyst) is added for reaction, whereby immobilization efficiency of the probe biomolecules and coating uniformity thereof are improved. Consequently, it is possible to dramatically improve quantitativity and reproducibility of the biosensor element.

### BACKGROUND OF THE INVENTION

The present invention relates to a method for manufacturing a biosensor element on the surface where nucleic acids, proteins, and the like are immobilized for sensing biomolecules and chemical reactions.

The human genome sequence has been entirely deciphered by the human genome project, and currently a subject matter of the study is shifting from the conventional “sequence analysis” to “functional analysis” that examines functions thereof. Data obtained from this functional analysis are considered to be able to provide a significant clue to elucidate life phenomena, and it is expected that the functional analysis may become a key to solve a problem in every field associated with living things, such as medical practice, environment, and foods.

In the functional analysis above, it is demanded that a gene having an enormous amount of information and a protein made from the gene be analyzed exhaustively and rapidly. Considering the above situation, a biochip has been developed, which is a kind of a biosensor element, typified by DNA microarrays and protein chips.

There are mainly two methods to manufacture the biochips. One is a method in which an amino acid or a nucleic acid base is chemically bonded sequentially one by one on a substrate, by means of photolithography or ink-jet, whereby probe biomolecules such as proteins or on-strand DNAs (deoxyribonucleic acid) are synthesized on the substrate in-situ (see U.S. Pat. No. 5,424,186, referred to as “patent document 1”). The other is a method in which the probe biomolecules are synthesized ex-situ, and subsequently immobilized on the substrate (U.S. Pat. No. 5,700,637, referred to as “patent document 2”).

It is expected that the biochip will be used in the future, for example, in medical diagnosis such as diagnosing cancer. If the biochip is used in the medical diagnosis, it is necessary that data obtained from the biochip have a high reliability. In the method where the probe biomolecules are synthesized on the substrate in-situ by means of photolithography or ink-jet, if there is an error in type of the nucleic acid base and/or the amino acid to be synthesized, or any defect occurs therein, it is impossible under existing circumstances to examine such error or defect, and remove those parts after being manufactured. Furthermore, an enormous production cost is required to obtain a long DNA and/or protein.

On the other hand, in the method where DNAs and proteins are synthesized ex-situ and then immobilized on the substrate, a refining process of the synthesized biomolecules can be performed after synthesis. Therefore, it is possible to remove biomolecules having a defect or the like in advance. Accordingly, probe biomolecules of high degree of purity can be immobilized on the surface, thereby enabling manufacture of a highly reliable chip. In many instances in this method, biomolecules having reactive groups react with the surface, and the immobilization is performed by forming a covalent bond. At this stage, photochemical reaction may be used.

However, a chip obtained by immobilizing on the substrate surface, a polymer of higher molecular weight, such as DNAs and proteins, may be easily subjected to an uncontrollably great deal of variations in amount and/or structure of the probe biomolecules being immobilized, resulting in that these variations may reduce reproducibility of data (Nature Vol. 21, pp. 5-9, 1999). In addition, when biomolecules are detected by use of the chip, the biomolecules may be non-specifically adsorbed in the substrate surface; the non-specifically adsorbed biomolecules may lower the S/N ratio of the detection. Furthermore, the low density of the probe biomolecules being immobilized may cause low sensitivity. These render quantitative analysis difficult (Nature Biotech. 19, p. 342, 2001).

The following three points are major causes of the above problems:
1) Reaction efficiency in immobilization is low, when the probe biomolecules synthesized ex-situ are immobilized on the chip surface;
2) Reaction active site on the chip surface is not uniform; and
3) Non-specifically adsorbed biomolecules remain on the surface.

One of the causes of 1) above is that while the substrate surface is hydrophobic in many cases, the probe biomolecule is comparatively hydrophilic, and thus affinity between the surface and the biomolecules is low. One of the causes of 2) above is that the coating film for producing the reaction active sites on the substrate surface is not uniform. One of the causes of 3) above is that non-specifically adsorbed biomolecules adhere to the surface and are hard to remove.

### SUMMARY OF THE INVENTION

The problems described above can be solved by providing a novel coating method which allows probe biomolecules to be immobilized efficiently and uniformly, and which further suppresses a non-specific adsorption of the probe biomolecules. In particular, it is considered that the problems can be solved by providing a coating method, in which 1) the affinity between the probe biomolecules and the substrate surface is increased, 2) the reaction active sites of the surface are made uniform, and 3) the non-specifically adsorbed biomolecules are hard to adhere to the surface and are easily removed therefrom.

Rickman et al., have been conducting a study of coating reaction solution used for uniformly immobilizing probe cDNAs, when the probe cDNAs are immobilized by a photochemical reaction with UV light. As a result of the study, Rickman et al., have reported that when a solution obtained by adding 3-[(3-cholamidopropyl)-dimethylammonio]-1-propane sulfonate in formamide or dimethyl sulfoxide (DMSO) solution, as solution for allowing the probe cDNAs to react is used, the immobilized amount is increased and also uniformity and reproducibility are improved (Nucleic Acids Research Vol. 31, No. 18 e109 (2003))

Rickman et al. employed UV light for immobilization, but when the UV light is used, there is a concern that the probe DNAs may be greatly damaged, thereby producing degenerative changes in the probe DNAs. Therefore, it is desirable that immobilization is performed without using the UV light. The present invention provides a method which immobilizes probe biomolecules efficiently and uniformly without deteriorating quality of the biomolecules.

The first aspect of the present invention provides a method for manufacturing a biosensor element where probe biomolecules for detecting a biochemical reaction are immobilized on a substrate, wherein a surfactant is added for reaction when the probe biomolecules are immobilized on the substrate.

In the second aspect of the present invention, if the probe biomolecules are nucleic acids, the surfactant is a positively charged surfactant.

In the third aspect of the present invention, if the probe biomolecules are proteins, and the effective charge of the protein molecules is negative, the positively charged surfactant is used as the surfactant, whereas a negatively charged surfactant is used as the surfactant if the effective charge of the protein molecules is positive.

In the fourth aspect of the present invention, the concentration C of the surfactant is 0.1 CMC≦C≦100 CMC (CMC: critical micelle concentration).

In the fifth aspect of the present invention, the concentration C of the surfactant is at least 1 CMC (CMC: critical micelle concentration).

In the sixth aspect of the present invention, when the probe biomolecules are immobilized in the biosensor element and the biomolecules are immobilized through the intermediary of reactive groups-containing silane coupling agent molecules, the water content of the reaction solution when the silane coupling agents are allowed to react with the substrate surface is at least 10%, and the reaction time is equal to or less than one hour.

As thus described, in the present invention, it is possible to immobilize biomolecules serving as a probe of the biosensor element on the substrate, efficiently and uniformly, and it is further possible to reduce the amount of non-specifically adsorbed biomolecules.

Therefore, sensitivity of the biosensor element can be largely improved, and further, it is also possible to enhance quantitativity and reproducibility of the biosensor element.

Accordingly, it is allowed to conduct a highly accurate/reliable gene/protein inspection and the like, by means of a small amount of inspection sample.

### BRIEF DESCRIPTION OF THE DRAWING

These and other features, objects and advantages of the present invention will become more apparent from the following description when taken in conjunction with the accompanying drawings wherein:

FIG. 1 is a procedural flow chart to explain a process for manufacturing a biosensor element.

FIG. 2 includes conceptual diagrams indicated by numerals (1), (2), and (3) for explaining a state of a substrate surface, respectively showing (1) a state being aminated (the first layer), (2) a state where reactive groups are introduced (the second layer), and (3) a state where probe biomolecules are immobilized.

FIG. 3 includes state diagrams indicated by numerals (1), (2), and (3) for specifically explaining the conceptual diagrams as shown in FIG. 2, respectively showing (1) a state being aminated (the first layer), (2) a state where reactive groups are introduced (the second layer), and (3) a state where probe biomolecules are immobilized.

FIG. 4A and FIG. 4B are schematic diagrams which explain a contribution of surfactant to the reaction when probe DNAs are immobilized.

FIG. 5 is a chart showing a relationship between CTAB (Cetyltrimethylammonium bromide) concentration in the reaction solution when the probe DNAs are immobilized, and DNA amount being immobilized.

FIG. 6 is a chart which shows the CTAB concentration in the reaction solution when the probe DNAs are immobilized, and in-array uniformity of the immobilized probe DNAs.

FIG. 7 is a chart showing a relationship between CTAB concentration in the reaction solution when the probe DNAs are immobilized, and the ratio of DNA which has been specifically adsorbed.

FIG. 8A and 8B are illustrations showing spotting shapes when the probe DNAs are immobilized.

FIG. 9 is a chart showing a relationship between hybridization amount and the CTAB concentration in the reaction solution when the probe DNAs are immobilized.

FIG. 10 is a schematic block diagram showing a structure of the probe DNAs immobilized on the substrate.

FIG. 11 is a schematic diagram showing a bead array.

### DETAILED DESCRIPTION OF PREFERRED EMBODIMENTS

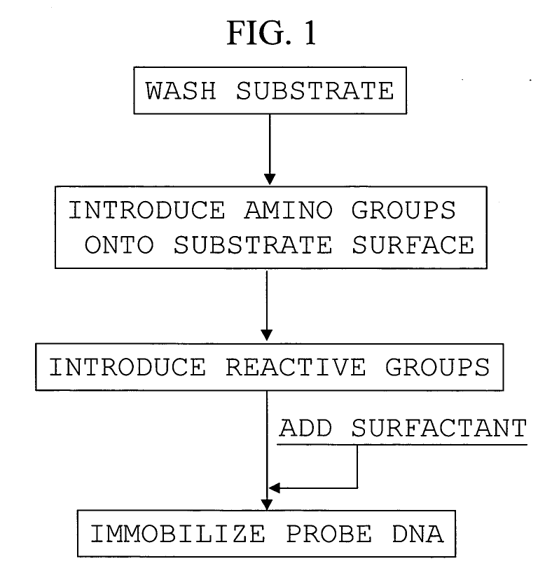

FIG. 1 is a process chart showing a method for manufacturing a biosensor element to which one embodiment of the present invention has been applied. The method for manufacturing the biosensor element according to the present embodiment, includes;

(1) a process for washing a carrier (substrate, beads, or the like),

(2) a process for introducing amino groups onto the surface of the carrier,

(3) a process for introducing reactive groups, and

(4) a process for immobilizing probe DNAs by use of a surfactant.

Hereinafter, each of the above processes will be explained as the following.

(1) Process for Washing a Carrier

A carrier according to a purpose is prepared and washed. Specifically, for instance, the carrier is washed with an alkaline aqueous solution such as NaOH aqueous solution, and then it is washed with an acid aqueous solution such as HCl aqueous solution, rinsed with purified water, and subsequently, it is dried under reduced-pressure.

For example, a glass substrate (slide glass), quartz substrate, plastic substrate, or the like can be used as the carrier. In addition, a metal coating substrate or the like may be available for the carrier. It is preferable that a material of the carrier is one having silanol groups on the surface.

It is not necessary that the carrier is a plane type. For example, the carrier may be in a form of beads, fiber, powder, or the like. When the carrier is in the form of beads, plastic beads such as polystyrene, metal coating beads, magnetic beads, or the like may be employed. As the cleaning solution, a mixed solution of sulphuric acid and hydrogen peroxide may be used.

(2) Process for Introducing Amino Groups

Silane coupling agents having amino groups are allowed to react with the carrier surface after the carrier surface has been washed, and the amino groups are immobilized on the carrier surface.

As the silane coupling agents, for instance, 3-aminopropyltrimethoxysilane, 3-aminopropyltriethoxysilane, N-(2-aminoethyl)-3-aminopropyltrimethoxysilane), (aminoethyl-aminomethyl) phenethyltrimethoxysilane, or the like may be used.

Ethanol, methanol, toluene, water, or the like can be used as the solvent, for instance. The reaction temperature is generally, in the range of 25° C.-85° C.

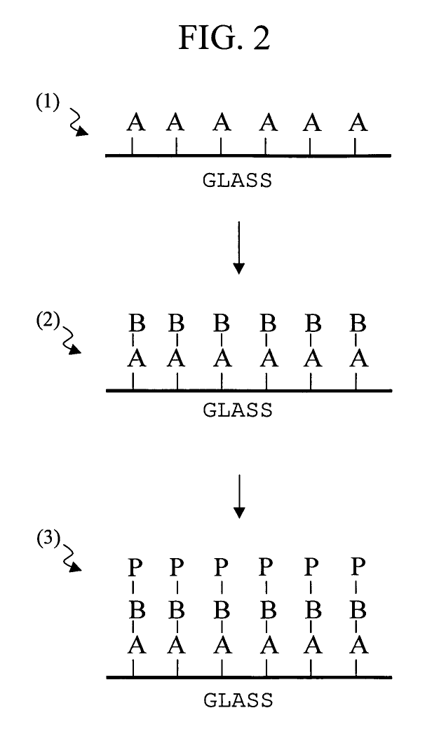

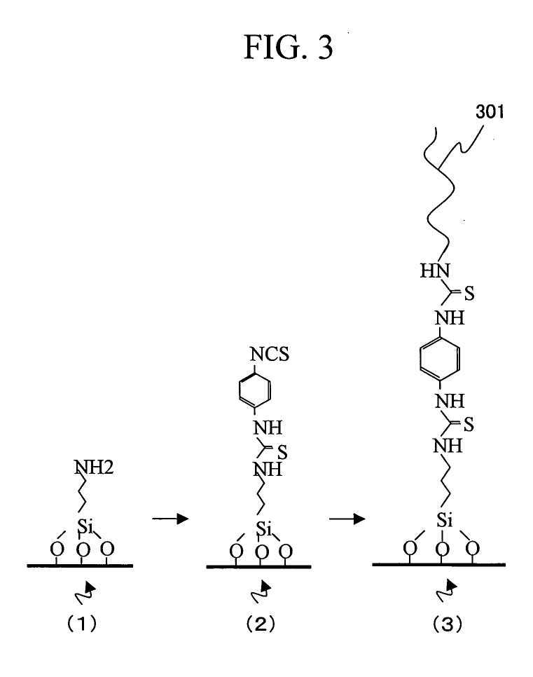

In FIG. 2, numeral (1) shows a state of the carrier surface (which is a glass in FIG. 2), where code “A” indicates a molecule immobilized on the first layer according to the above process. There exists an amino group on the terminal of A. In FIG. 3, numeral (1) shows 3-aminopropyl triethoxysilane that is used as the silane coupling agent.

(3) Process for Introducing Reactive Groups

A compound having reactive groups is allowed to react with the amino groups on the carrier surface, and the reactive groups are immobilized on the carrier surface.

As the compound having reactive groups, PDC (Phenylenediisothiocyanate) having isothiocyanate groups on the terminal, or the like, DSG (Disuccinimidyl glutarate) having succinimide groups, or the like, KMUS (N-(11-maleimidoundecanoyloxy) succinimide) having maleimide groups, or the like can be employed. Here, the terminal isothiocyanate groups, succinimide groups, or maleimide groups are referred to as “reactive groups”.

DMF (N,N-Dimethyolformamide), DMSO (DimethylSulfoxide), ethanol, pyridine, or the like can be used as the solvent, for instance. The reaction temperature is generally in the range of 25°C.-85° C.

In FIG. 2, numeral (2) shows a state of the carrier surface, where code “B” indicates a part of the reactive group immobilized on the second layer according to the above process. In FIG. 3, numeral (2) shows the PDC that is used as a compound having the reactive groups.

(4) Immobilization of Probe DNA

The reactive groups formed in the carrier surface are allowed to react with probe DNAs; the terminal of the probe DNAs has a group that is capable of reacting with the reactive groups formed on the carrier surface, whereby the probe DNAs are immobilized on the carrier surface. At this timing, in order to enhance the reactive efficiency of the immobilization of the probe DNAs, a surfactant is added in the reaction solution.

The surfactant to be added, which works as a phase transfer catalyst, will be explained. For a two-phase system, water phase and oil phase, the surfactant accelerates transferring of ions between two phases, the ions dissolved in the water phase and ions dissolved in the oil phase. In other words, the frequency of the collision between molecules dissolved in respective phases is increased, thereby enhancing efficiency of the reaction between two phases.

Though there are two types of surfactant, nonionic and ionic, it is preferable to use an ionic surfactant. It is further preferable to use a positively charged surfactant.

Such a surfactant as mentioned above may include, CTAB(Cetyltrimethylammonium bromide) which is also a transfer catalyst; CₙH₂ₙ₊₁NH₃(Cl⁻) or CₙH₂ₙ₊₁NH₃(Br⁻) (n is at least 8), which is an ammonium salt; CₙH₂ₙ₊₁N(CH₃)₃(Cl⁻) or CₙH₂ₙ₊₁N(CH₃)₃(Br⁻) (n is at least 8); CₙH₂ₙ₊₁N(C₃H₇)₃(Br⁻) (n is at least 14); C₁₈H₃₇N(CH₃)₃(NO₃⁻), C₁₈H₃₇N (CH₃)₃(FO₃⁻), C₁₈H₃₇N(C₂H₅)₃(BrO₃⁻), C₁₈H₃₇N(C₃H₇)₃(BrO₃⁻), C₁₈H₃₇N(C₄H₉)₃(BrO₃⁻); or other than above, tetraalkylammonium salt, trialkylphenylammonium salt, trialkylbenzylammonium salt, alkylpyridinium halides, and the like are taken as examples. Any of the surfactants as listed above may be used alone, or may be used by mixing at least two types thereof.

It is preferable that the added amount of the surfactant is to provide a concentration around criticalmicelle concentration (referred to as “CMC”) Here, the “critical micelle concentration” will be explained. The critical micelle concentration is a concentration where the correlation between the concentration of the surfactant and a surface tension of the solution becomes discontinuous, when the surfactant is added to the solution. When the concentration is equal to or less than the critical micelle concentration, the surfactant molecules exist in a form of a monomer in the solution or on the surface of the carrier (substrate). On the other hand, within the region of the critical micelle concentration, the surfactant molecules become an aggregate to form a lump (micelle). Further on the carrier (substrate), the surfactant molecules form a monolayer. As thus described, behavior of the surfactant molecules is changed drastically around the critical micelle concentration.

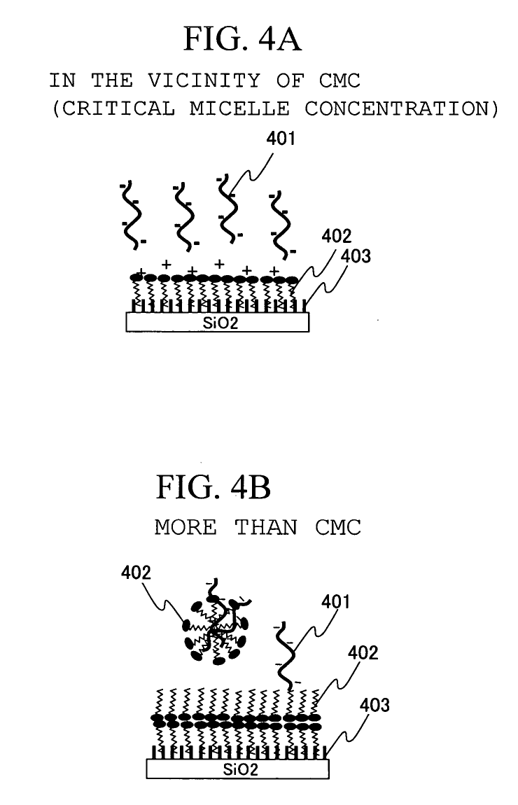

FIG. 4A shows a mechanism for how the amount of the immobilized probe DNAs is increased, in the case where a positively charged surfactant is added at a concentration that is in the vicinity of the critical micelle concentration. In the vicinity of the critical micelle concentration, the positively charged surfactant 402 is adsorbed as a single layer on the reactive groups 403, and the negatively charged probe DNA 401 is captured with Coulomb force. The surfactant 402 is a phase transfer catalyst, and it improves affinity between the hydrophobic part and the hydrophilic part. In other words, the concentration of the probe DNAs adjacent to the carrier surface can be increased, and further the collision frequency between the probe DNA (hydrophilic part) and the reactive group on the substrate (hydrophobic part) can be increased.

On the other hand, as shown in FIG. 4B, when the concentration is more than the critical micelle concentration, multilayered adsorption of the surfactant and/or creation of a micelle may occur on the substrate surface. The adsorbed multilayer may block a diffusion of the probe DNA onto the surface, and the micelle may trap the probe DNAs in the fluid. Therefore, the multilayered adsorption of the surfactant and/or the micelle may work toward blocking the reaction between the probe DNAs and the reactive groups on the substrate.

However, the multilayer and the micelle are not rigidly structured, and are structured dynamically where association/dissociation is coexisting. Therefore, characteristics of the phase transfer catalysts are maintained. Therefore, although the reaction efficiency is down if compared with the condition where the concentration is around the critical micelle concentration, if compared with the condition where the surfactant is not added, the reaction efficiency is enhanced.

Furthermore, there is a feature that in the concentration region equal to or more than the critical micelle concentration, the reacting amount of the probe DNAs on the substrate surface is stable even if the concentration of the surfactant is changed. This feature is advantageous from the viewpoint of building a robust process.

As a specific concentration of the surfactant, it is desirable to add the surfactant in a concentration of at least 0.1 CMC (in the case of CTAB, 0.08 mM). If the concentration is equal to or more than 0.1 CMC, it is possible to reduce the ratio of non-specifically adsorbed probe DNAs. On the other hand, if the concentration is set to high, such as more than 100 CMC (for the case of CTAB, 80 mM), the reaction efficiency falls as described above. Furthermore, the wettability between the substrate surface and the reaction solution is extremely increased, and thus the spot shape may easily become a distorted form, instead of a desired symmetrically circular form. Therefore, it is desirable that the concentration is equal to or less than 100 CMC.

It is to be noted that the amount of solution which is spotted for immobilizing the probe DNAs may be around a few pL per spot. Even under highly humid circumstances, there is a case that moisture in the solution evaporates and the concentration of the surfactant may vary depending on the spot. For this case, it is desirable that the immobilized amount of probe DNAs is not varied drastically according to the change of the surfactant concentration.

The concentration region of the surfactant, which may not drastically change the immobilized amount of probe DNAs, is at least 1 CMC (for the case of CTAB, 0.8 mM), and preferably, at least 10 CMC (for the case of CTAB, 8 mM). In other words, it is desirable to add the surfactant within this concentration range, in order to immobilize the probe DNAs in a range with little change according to the concentration change of the surfactant, to improve the reaction efficiency, and to reduce the amount of probe DNAs which are adsorbed non-specifically.

The solution in which the probe DNAs are to be dissolved may include a weakly alkaline aqueous solution, such as a carbonic acid buffer and a phosphoric acid buffer. Then, the probe DNAs are dissolved into this solution, and further the surfactant is added, so as to obtain the reaction liquid.

If the carrier is a glass substrate, the reaction liquid in which the probe DNAs are dissolved may be spotted on the surface of the substrate.

If the carrier is in a form of beads, the beads may be soaked in the reaction liquid in which the probe DNAs are dissolved.

The reaction temperature is generally in the range from 25° C. to 40° C. The reaction time is in the range from 2 to 12 hours. The reaction is made to occur under the circumstances where humidity is maintained sufficiently, so that the solution may not be dried during the reaction.

With the processing as described above, when the reactive groups are isothiocyanate groups or succinimide groups, the probe DNA having an amino group on the 5′-terminal can be immobilized. Alternatively, if the reactive groups are maleimide groups, the probe DNA having a thiol group on the 5′-terminal can be immobilized.

In FIG. 2, numeral (3) shows a state of the carrier surface where “P” represents the probe DNA as described above. In FIG. 3, numeral (3) shows a case where the reactive groups are isothiocyanate groups and the probe DNA having the amino group on the 5′-terminal is immobilized.

Up to this point, one embodiment of the present invention has been applied to a method for manufacturing a biosensor element.

In the above embodiment, an explanation has been made focusing on the process to immobilize the prove DNAs. In the manufacturing steps above, however, another process may be performed to improve efficiency and uniformity of immobilization of lower layers of the probe DNA (layers corresponding to A and B in FIG. 2), in order that the probe DNAs can be immobilized efficiently and uniformly.

By way of example, in the process for introducing the amino groups, when the substrate surface is coated with the first layer (part A of FIG. 2), the water content of the reaction solution and the reaction time are adjusted appropriately.

Specifically, it is preferable to set the water content of the reaction solution containing silane coupling agents to 10% or more. Setting the water content of the reaction solution too small, such as less than 10%, may deteriorate the uniformity and reduce the volume of immobilized probe DNAs. In the range where the water content is large, such as 10% or more, the uniformity in immobilization of probe DNAs is improved, and the immobilized amount of DNA is increased.

The aforementioned results will be obtained due to the following reasons. When the silane coupling agents react with the surface, silanol groups of the agents react with the substrate surface; those silanol groups are generated by hydrolysis of methoxy groups, ethoxy groups, or the like of the silane coupling agents. If the substrate is made of glass, for instance, a siloxane bond (Si—O—Si) is formed. If the water content is small in amount, the silanol groups are hardly generated. Therefore, amino groups in the silane coupling agents, positively charged, may be dominantly adsorbed in the substrate surface due to the negative charge of the surface, and this may interfere with the reaction.

On the other hand, when the water content is large, the methoxy group part or the ethoxy group part in the silane coupling agents may be swiftly changed to the silanol groups via the hydrolysis reaction, and a reaction proceeds rapidly, in which a siloxane bond is formed by reacting the silanol groups and the surface of the substrate. In addition, the silanol polymerization reaction generates water, and thus if the water content in the reaction solution is large, the silanol polymerization reaction is suppressed. Accordingly, if the water content is large, amination can be performed uniformly, because of the synergistic effect as described above.

In the meantime, it is preferable that the reaction time between the carrier and the silane coupling agents is less than one hour. If the reaction time is one hour or more, the uniformity may be deteriorated and the immobilized amount may be reduced. A cause for the above result is considered to be that if the reaction time is long, the polymerization reaction among the silane coupling agents may proceed, and an aggregate as a product of the polymerization reaction may adhere on the substrate surface.

Consequently, in the process for introducing the amino groups, it is desirable to perform the process under the circumstances that the reaction time is less than one hour, and the water content of the reaction solution is 10% or more. From the viewpoint of actual operability, it is preferable to set the reaction time from one minute to one hour, and the water content of the reaction fluid is set to 10% to 50%.

In the above embodiment, DNA has been used as the biomolecule, but another biomolecule such as RNA (ribonucleic acid), protein, PNA, sugar chain, or a composite of these elements may also be available.

When a protein is employed, if the protein is negatively charged in the aqueous solution, it is desirable to use a positively charged surfactant as a surfactant to be added in the immobilization solution. On the other hand, if the protein is positively charged in the aqueous solution, it is desirable to use a negatively charged surfactant as a surfactant. The negatively charged surfactant may be, for instance, sulfate esters, sulfonate, alkyl benzene sulfonate, carboxylate, and the like. If the negatively charged surfactant as described above is used, probe immobilization efficiency and uniformity may be improved.

Furthermore, in the above embodiment, the amino groups are firstly introduced on the carrier surface by use of the silane coupling agents. However, similar effects can be obtained by adding the surfactant as described above, also in the case where the molecules having a carboxyl group, epoxy group, and the like are immobilized, or in the case where the active groups are immobilized with a coating of avidin.

By use of the immobilization method as described above, it is possible to form a chip surface where the density of the probe biomolecules is 2×10¹² molecule/cm² or more, and the ratio of the probe biomolecules which are non-specifically adsorbed and which are not bonded by forming a covalent bond, is 10% or less.

### EXAMPLES

Next, examples of the present invention will be explained in detail. It is to be noted, however, that the present invention is not limited to the following examples.

In the following, examples of the manufacturing method according to the present invention, which utilize a plane-type DNA microarray and a bead array, will be described.

### Example 1

### Immobilization of Linker Molecule (Molecule having Reactive Groups) onto a Substrate

A slide glass made of borosilicate glass was prepared as a carrier. According to the process as shown in FIG. 1, the substrate was washed in NaOH aqueous solution, further washed in HCl aqueous solution, and then, rinsed with purified water. Subsequently, it was dried under reduced pressure. 3-aminopropyltrimethoxysilane, which serves as the silane coupling agent, was allowed to react with the washed substrate surface, and the substrate surface was aminated (see FIG. 3, numeral (1)). In addition, methanol was used as the solvent, and the concentration of the silane coupling agent was 3% (Volume/Volume) The reaction temperature was room temperature, and the reaction time was five minutes.

Next, the aminated substrate was acted upon by PDC (Phenylene diisothiocyanate) having isothiocyanate groups on the terminal. Here, DMF(N,N-dimethylformamide)was used as the solvent, and the concentration of PDC was 0.6% (Weight/Volume) The reaction temperature was room temperature (around 15° C. to 30° C.), and the reaction time for that temperature was 12 hours. It is to be noted that any reaction temperature is applicable so far as it induces a reaction, and the range from 4° C. to 40° C. is sufficient. Accordingly, a substrate was obtained, on which the reactive groups (linker molecules) were immobilized (see FIG. 3, numeral (2)).

### Example 2

### Preparation of Reaction Liquid Containing Probe DNA

100 μM of 50-mer probe DNA having a sequence of 50 bases was dissolved in a weak alkaline carbonic acid buffer (1M Na₂CO₃ and 1M NaHCO₃ are mixed and adjusted to pH 9.0). This solution was preparatively isolated, and CTAB (Cetyltrimethylammonium bromide) serving as a positively charged surfactant, with various concentrations from 0 mM to 80 mM, was respectively added to thus isolated solutions.

It has been reported that the critical micelle concentration (CMC) of CTAB is 0.8 mM (Langumuir Vol. 1 No. 3, p. 352, 1985). Therefore, according to the above process, a solution has been obtained which was added with CTAB having a concentration close in range to the critical micelle concentration.

### Example 3

### Immobilization of Probe DNA onto the Substrate

On a plurality of substrates obtained in the Example 1, the reaction solutions prepared in Example 2, being different in concentration, were respectively spotted, and substrates with the probe DNA being immobilized were obtained. Here, the reaction temperature was 25° C., and the reaction time was four hours. The reaction was allowed to occur under the circumstances where humidity was maintained sufficiently, so that the solutions may not be dried during the reaction.

### Example 4

### Evaluation

In order to measure the immobilized amount of probe DNAs on the substrate that were obtained in Example 3, nucleic acid base (ddTTP-Cy5) with fluorescent dye being attached was modified on the 3′-terminal of the immobilized probe DNAs, by use of Terminal deoxynucleotidyl transferase, and the fluorescence intensity was measured by a fluorescence scanner, the intensity being obtained by exciting the fluorochrome cy5.

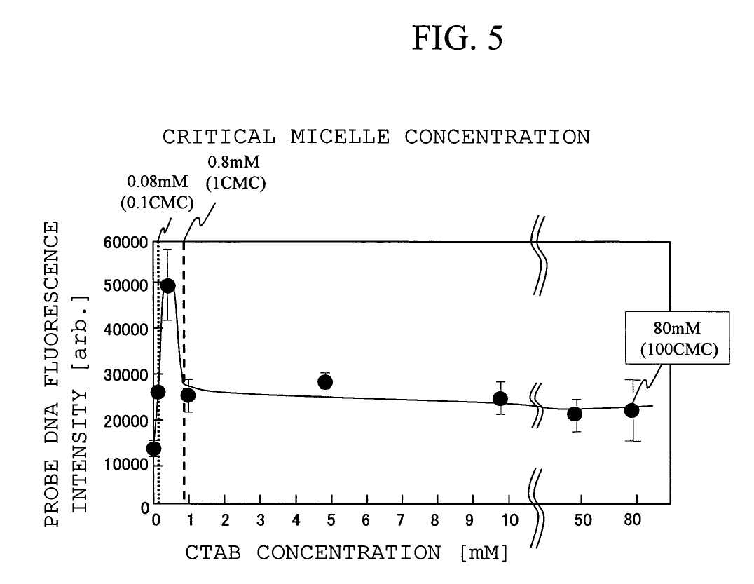

FIG. 5 shows a result of the above measurement. According to FIG. 5, it is found that the average amount of immobilized probe DNAs is increased in proportion to the added amount of CTAB. When the concentration becomes around 0.8 mM being the critical micelle concentration (CMC), the average amount of immobilized probe DNAs is almost maximized and becomes approximately by four times compared to the case where the CTAB is not added. If the concentration of CTAB is increased further, the immobilized amount goes down.

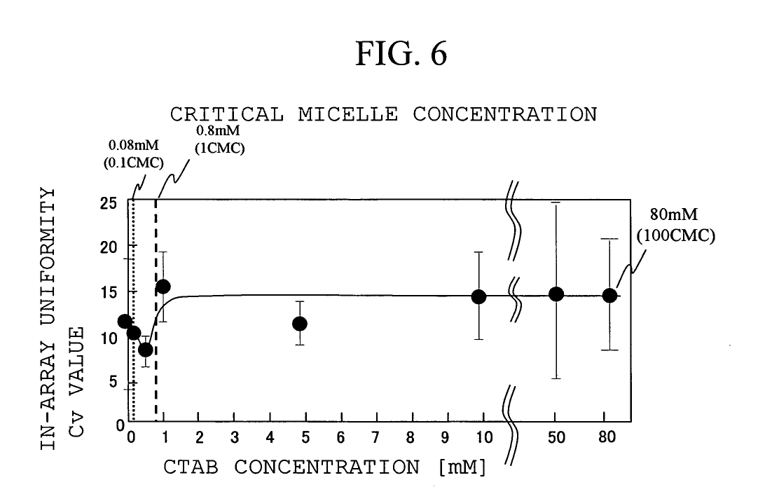

FIG. 6 shows a relationship between uniformity in probe DNA immobilization and CTAB addition concentration. The uniformity is expressed by a value of Cv (Coefficient of Variation) of the fluorescence intensity among a plurality of spots. It is found that when the CTAB addition concentration is around the CMC, the uniformity is enhanced. Therefore, it is further understood that adding CTAB with an appropriate concentration is effective for the uniformity of the probe DNAs.

Furthermore, the substrate obtained in Example 3 was used to measure the probe DNA amount which was non-specifically adsorbed when the CTAB was added. The Probe DNAs having amino groups on the terminal react with the reactive groups on the substrate surface to form a covalent bond, so as to be immobilized, but the probe DNA which does not have amino groups on the terminal does not form the covalent bond. Therefore, the attached amount of probe DNAs that do not have amino groups is equal to the amount non-specifically adsorbed. Here, a ratio of the non-specifically adsorbed amount is calculated. The ratio of the non-specifically adsorbed amount is obtained by use of the aforementioned fluorochrome (cy5), and it is the ratio of the immobilized amount of probe DNAs without amino groups on the terminal, to the immobilized amount of probe DNAs having amino groups on 5′-terminal. The probe DNAs used for the experiment have the same base sequence of 50-mer probe DNA.

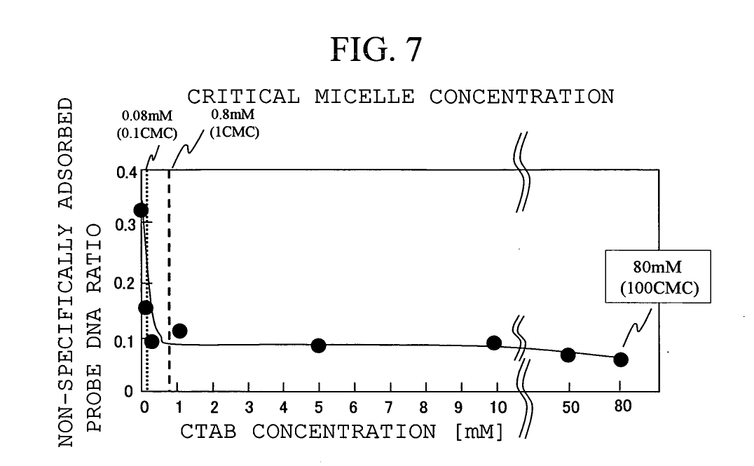

FIG. 7 shows a relationship between CTAB concentration and the ratio of non-specifically adsorbed amount. It is found that by adding the CTAB, the ratio of non-specifically adsorbed probe DNAs can be reduced to 10% or less.

It is found according to the results so far that in order to improve the reaction efficiency by approximately twice or more to immobilize the probe DNAs onto the substrate surface, it is desirable to add the CTAB with the concentration in the range of 0.1 CMC (0.08 mM) or more. As shown in FIG. 7, if the concentration is 0.1 CMC or more, it is also possible to reduce the ratio of probe DNAs which are non-specifically adsorbed.

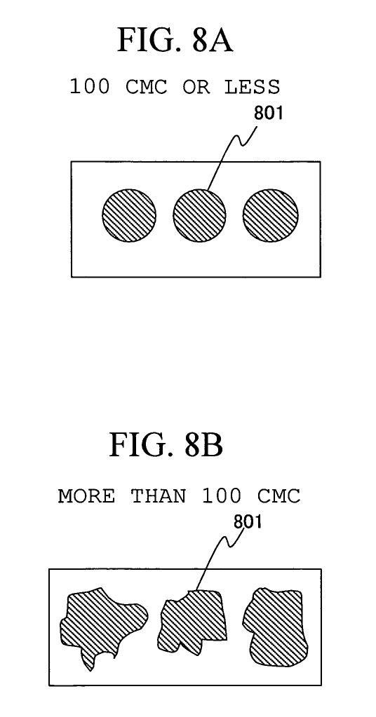

On the other hand, if the CTAB concentration is set to high, such as more than 100 CMC (80 mM), the wettability between the substrate surface and the reaction solution is extremely increased, and the spot shape may easily become distorted as shown in FIG. 8B, instead of symmetrically circular. Therefore, it is found desirable that the CTAB concentration is set to 100 CMC or less.

The amount of solution spotted for immobilizing the probe DNAs is around a few pL per spot, in the case of a small amount. Even under the highly humid circumstances, moisture in the solution may evaporate and the CTAB concentrations may vary by spot. It is desirable in the above case that the immobilized probe DNA amount may not change drastically in response to the change of CTAB concentration.

According to FIG. 5, a range of CTAB concentration that may not cause a drastic change of probe DNA immobilized amount is at least 1 CMC (0.8 mM), and more preferably, it is 10 CMC (8 mM) or more. Consequently, it is found that addition of CTAB with the concentration above is desirable, in order to immobilize the probe DNAs within the range where the immobilized amount may not be changed so much depending on the CTAB concentration, to enhance the reaction efficiency, and further to reduce the probe DNAs adsorbed non-specifically.

### Example 5

### Hybridization

The quantity of Hybridization as to the immobilized probe DNAs was evaluated. A target DNA was hybridized on the substrate on which the probe DNAs were immobilized by use of the solution in which CTAB with concentration of 0.1 CMC≦C≦100 CMC was added according to Example 1 to Example 3, the target DNA being completely complementary with the probe DNA. 5×SSC (Standard Saline Citrate), 0.5% SDS solution (Sodium Dodecylsulphate) was used as the hybridization solution, and after the hybridization at 42° C., washing was performed with 2×SSC, 0.1% SDS solution and 1×SSC, 0.1% SDS solution. The terminal of the complete complementary target DNA is modified with a fluorescent molecule of Cy5.

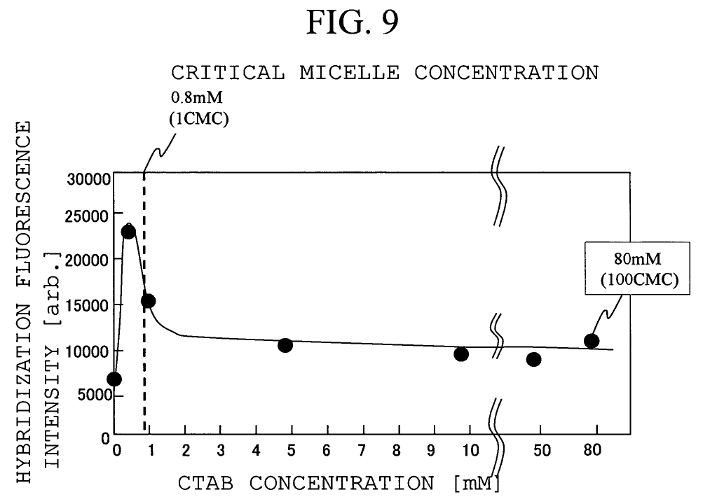

FIG. 9 shows a result of measured fluorescence intensity by use of the fluorescent scanner, after hybridization. It is found that by immobilizing the probe DNAs with the solution in which CTAB is added, the fluorescence intensity is increased after hybridization.

### Example 6

### Measurement of Probe DNA Density

Here, a result of detailed examination is shown as to the density of the probe DNAs which were immobilized by Example 3. The adherent amount of the probe DNAs was measured by use of X-ray reflectance, and the result was that the probe DNA density was equal to or more than 2×10¹² molecule/cm², when at least 0.1 CMC of CTAB was added.

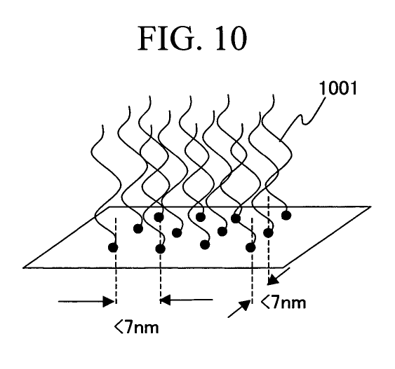

FIG. 10 is a schematic block diagram showing the substrate surface when the density of the probe DNA 901 is 2×10¹² molecule/cm². It is found that by adding CTAB of at least 0.1 CMC, the density of probe DNAs is allowed to be equal to or more than 2×10¹² molecule/cm², that is, the distance between the two probe DNAs is allowed to be within 7 nm.

### Example 7

### Influences of Water Content in Reaction Solution and Reaction Time, in the Process of Introducing Amino Groups

Influences of the water content in the reaction solution and the reaction time, when amino groups are introduced in Example 1, were examined. Table 1 shows a result of the examination as to the relationship as to the water content of the reaction solution, reaction time, the probe DNA uniformity, and its immobilized amount.

The probe DNA uniformity is expressed in Cv value of fluorescence intensity among spots within the array. When the water content in the reaction solution is less than 10%, the uniformity is deteriorated, and the immobilized amount is reduced. However, when the water content is in the range of a large amount such as 10% or more, the immobilization uniformity of the probe DNAs is improved, and the amount of DNA having been immobilized is large. As for the reaction time, it has been found that if the reaction time is one hour or more, the uniformity is deteriorated, and the immobilized amount is reduced.

Consequently, it is found desirable to perform amination with the reaction time of less than one hour, and with at least 10% water content of the reaction solution.

TABLE 1
IMMOBILIZED AMOUNT AND UNIFORMITY OF PROBE DNAS
ACCORDING TO SILANE COUPLING REACTION CONDITIONS

| | | | PROBE DNA | | |
|---|---|---|---|---|---|
| | | | IMMOBILIZED | | |
| WATER | | | AMOUNT | PROBE DNA | |
| CONTENT | REACTION | | FLUORESCENCE | IMMOBILIZED | |
| FOR | TIME FOR | | INTENSITY | UNIFORMITY | |
| AMINATION | AMINATION | | [×10³ arb.] | [%] | JUDGMENT |
| 0% | 5 | min | 5.2 | 14 | |
| | 30 | min | 5.0 | 14 | |
| | 1 | hr | 4.9 | 15 | |
| | 5 | hr | 3.9 | 17 | |
| 2% | 5 | min | 6.2 | 13 | |
| | 30 | min | 5.7 | 13 | |
| | 1 | hr | 5.5 | 14 | |
| | 5 | hr | 5.0 | 15 | |
| 10% | 5 | min | 12.5 | 12 | ◯ |
| | 30 | min | 11.7 | 12 | ◯ |
| | 1 | hr | 10.5 | 14 | |
| | 5 | hr | 9.5 | 15 | |
| 20% | 5 | min | 15.2 | 11 | ◯ |
| | 30 | min | 14.0 | 12 | ◯ |
| | 1 | hr | 13.1 | 13 | |
| | 5 | hr | 12.5 | 14 | |

### Example 8

In order to manufacture a bead array for genetic analysis use, beads were employed instead of the substrate, and the beads on which probe DNAs were immobilized by Examples 1 to 3 were obtained. In addition, multiple types of beads were obtained by immobilizing probe DNAs having one base sequence per bead. The probe DNAs were immobilized after the beads were soaked in the reaction solution. A material of the beads used here were borosilicate glass, and the bead diameter was around 100 μm.

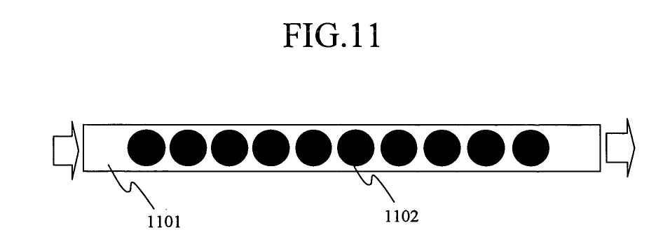

FIG. 11 shows a bead array for genetic analysis use, where 10 types of beads 1002 on which probe DNAs that are different from one another are immobilized, being manufactured according to the above process, and are accommodated in a micro channel 1001, thereby forming one array.

As for the beads as described above, similar to the results as shown in FIG. 5, FIG. 6, and FIG. 7, it was possible to improve the probe DNA immobilization efficiency and to enhance the uniformity, by adding a positively charged surfactant as a phase transfer catalyst.

Furthermore, it was possible to reduce the non-specific adsorbed amount of probe DNAs. When the reaction time for the amination was set to within 1 hour, and the reaction solution water content was set to 10% or more, the probe DNA immobilization efficiency and uniformity were improved, similar to the results as shown in Table 1.

With the conditions above, probe DNAs were immobilized on the beads, and target DNAs having Cy5 as a fluorescent molecule were hybridized, the target DNA being completely complementary with the probe DNA. At this stage, the fluorescence intensity and uniformity were examined. The fluorescence intensity was measured by means of a fluorescence scanner. As a comparative example, the reaction time was set to 5 hours and water content was set to 2% as conditions for amination, and probe DNAs were immobilized on the beads under a condition that a surfactant was not added. Then, beads obtained according to the above process were also employed.

Consequently, the case where the beads on which probe DNAs were immobilized under the conditions according to the present invention shows higher fluorescence intensity after hybridization. This means that the target DNAs can be captured on the beads surface more efficiently. In addition, it is found that variation in fluorescence intensity between beads on which the same probe DNAs have been immobilized can be reduced.

While we have shown and described several embodiments in accordance with our invention, it should be understood that disclosed embodiments are susceptible of changes and modifications without departing from the scope of the invention. Therefore, we do not intend to be bound by the details shown and described herein but intend to cover all such changes and modifications that fall within the ambit of the appended claims.

What is claimed is:

1. A method for manufacturing a biosensor element in which probe biomolecules are immobilized on a substrate, wherein a surfactant is added in a reaction solution for immobilization, when the probe biomolecules are immobilized on the substrate.

2. The method for manufacturing a biosensor element according to claim 1, wherein,
said probe biomolecules are nucleic acid, and said surfactant is positively charged surfactant.

3. The method for manufacturing a biosensor element according to claim 1, wherein,
when said probe biomolecules are protein and effective charge of the protein molecules is negative, positively charged surfactant is used as said surfactant, whereas negatively charged surfactant is used as said surfactant when the effective charge is positive.

4. The method for manufacturing a biosensor element according to claim 1, wherein,
concentration C of said surfactant is 0.1 CMC≦C≦100 CMC (CMC: critical micelle concentration).

5. The method for manufacturing a biosensor element according to claim 1, wherein,
concentration C of said surfactant is at least 1 CMC (CMC: critical micelle concentration).

6. The method for manufacturing a biosensor element according to claim 1, wherein,
when said probe biomolecules are immobilized and said biomolecules are immobilized through the intermediary of silane coupling agent molecules, water content of the reaction solution when the silane coupling agents are allowed to react with the substrate surface is at least 10%, and the reaction time is equal to or less than one hour.

7. A biosensor element in which probe biomolecules are immobilized on a substrate, wherein a surfactant is added in a reaction solution for immobilization, when the probe biomolecules are immobilized on the substrate.

8. The biosensor element according to claim 7, wherein,
a density of the probe biomolecules is 2×10¹² molecule/cm² or more.

9. The biosensor element according to claim 7, wherein,
a ratio of the probe biomolecules non-specifically adsorbed is 10% or less.

10. The biosensor element according to claim 7, wherein,
said probe biomolecules are nucleic acid, and said surfactant is positively charged surfactant.

11. The biosensor element according to claim 7, wherein,
when the probe biomolecules are protein, and effective charge of the protein molecules is negative, positively charged surfactant is used as said surfactant, whereas negatively charged surfactant is used as said surfactant when the effective charge is positive.

12. The biosensor element according to claim 7, wherein,
concentration C of said surfactant is 0.1 CMC≦C≦100 CMC (CMC: critical micelle concentration).

13. The biosensor element according to claim 7, wherein,
concentration C of said surfactant is at least 1 CMC (CMC: critical micelle concentration).

14. The biosensor element according to claim 7, wherein,
when said probe biomolecules are immobilized and said biomolecules are immobilized through the intermediary of silane coupling agent molecules, water content of the reaction solution when the silane coupling agents are allowed to react with the substrate surface is at least 10%, and the reaction time is equal to or less than one hour.
